(19) United States
(12) Patent Application Publication
(10) Pub. No.: US 2023/0151416 A1
(43) Pub. Date: May 18, 2023

(54) TEST PLATE AND AUTOMATED BIOLOGICAL TEST SYSTEM

(71) Applicant: WITHINGS, ISSY LES MOULINEAUX (FR)

(72) Inventors: Benoît TUCOULAT, ISSY LES MOULINEAUX (FR); Julius DEWAVRIN, ISSY LES MOULINEAUX (FR); Victor TING, ISSY LES MOULINEAUX (FR); Christelle BARAKAT, ISSY LES MOULINEAUX (FR); Sounderya NAGARAJAN, ISSY LES MOULINEAUX (FR)

(21) Appl. No.: 17/919,429

(22) PCT Filed: Mar. 26, 2021
(86) PCT No.: PCT/FR2021/050545

§ 371 Date: Oct. 17, 2022

(30) Foreign Application Priority Data
Apr. 28, 2020 (FR) FR20 04218

(51) Int. Cl. C12Q 1/6844; B01L 3/00

(52) U.S. Cl. CPC C12Q 1/6844; B01L 3/502715; B01L 3/502738; B01L 3/50273; B01L 2300/044; B01L 2200/027; B01L 2200/0684; B01L 2200/10; B01L 2300/02; B01L 2300/0672; B01L 2300/0861; B01L 2300/1805; B01L 2300/0816; B01L 2400/0638; B01L 2300/042; B01L 2400/0487; B01L 2300/0663; B01L 2200/0689

(57) ABSTRACT

A single-use test plate in a biological test system for determining the presence of one or more nucleic acid sequences, the test plate including a body having channels and sockets formed therein, an inlet for receiving a test sample, an air inlet, an extraction membrane, reaction zones containing a porous media with first and second reaction mixtures allowing amplification and detection of the nucleic acid species, an air outlet, a recovery reservoir, the plate being configured to feed the biological sample to be tested into the extraction membrane, then after rinsing, drying and elution of said extraction membrane, to feed the resulting eluate into the reaction zones, in order, after reaction, to deduce therefrom a result through the readout zone.

The present document relates generally to diagnostic devices by nucleic acid amplification, in particular, from a biological sample such as drops of blood, urine, saliva and sweat. More specifically, the present invention relates to test plates, used in an automated biological test system for identifying the presence of an infectious microorganism (bacteria, virus, etc.). However, the proposed device and method may be applied to the detection of other biological markers that may reveal any other type of pathology, including cancer.

For diagnostic tests based on the detection of DNA/RNA of an infectious microorganism by amplification, the usual PCR tests (PCR for Polymerase Chain Reaction) requiring temperature cycles are known, which are long and costly to implement (requiring the use of expensive thermal cyclers).

In more rapid diagnostic tests, a type of test known as LAMP (Loop-mediated isothermal amplification) or RT-LAMP (Reverse transcriptase Loop-mediated isothermal amplification) has recently been used, for which it is no longer necessary to perform several temperature cycles. A temperature plateau is sufficient, hence the term “isothermal”.

In this context, a need has arisen for a biological test system that can process a large number of samples reliably and quickly, and that is suitable for an isothermal type of test, without excluding its application to other types of tests.

To this end, a test plate is proposed for single use in a biological test system for detecting the presence of one or more pathogenic species (e.g., a nucleic acid sequence of interest), the test plate comprising:

a body in which channels and housings are formed, which are generally isolated from the external environment,
an inlet for receiving a biological sample to be tested in liquid form, the inlet preferably being sealable or reclosable,
optionally at least one air inlet,
an extraction membrane, arranged in a first housing,
a first reaction zone containing a porous media arranged in a second housing and containing a first test reaction mixture in lyophilized form, the first reaction mixture allowing the amplification and detection of at least one nucleic acid sequence of interest,
preferably at least one second reaction zone containing a porous media arranged in a third housing and containing a second control reaction mixture (positive or negative) in lyophilized form,
optionally at least one air outlet,
optionally a recovery reservoir,
a result reading zone, comprising at least the first reaction zone and, if applicable, the second reaction zone, the test plate being configured to feed the biological sample to be tested into the extraction membrane and then, after rinsing, drying and eluting said extraction membrane, to feed the resulting eluate into the reaction zones, in order, after reaction, to deduce a result through the reading zone.

Thanks to the above provisions, the test process may be automated and secured. The test plate is inserted into an analysis station which carries out actions corresponding to the above process. In particular, the drying of the extraction membrane is done in situ by an air flow that circulates in the channels and the housing that contains the extraction membrane, thanks to the air inlets/outlets. Alternatively, the membrane may be dried by a heating device. No manipulation is required during the test, the plate remains in the analysis station, everything takes place under the control of the station, the latter preferably having a closed cover during the test. The membrane is quickly and correctly dried, which increases the reliability and speed of the test.

The term “extraction membrane” is used here to refer to a porous member that momentarily absorbs nucleic acids from the sample to be tested, retains the nucleic acids during one or more rinses to remove chemical species such as proteins or debris, including lysis buffer residues, whereupon an eluent with a higher ionic affinity to said nucleic acids is used to capture the nucleic acids and carry them to the reaction/amplification zones.

The term “plate” must be taken here in a very broad sense, the plate is here a support which may take any geometrical form.

It should be noted that the second reaction zone, known as the control zone, makes it possible to confirm that the reaction has been carried out correctly. The second control reaction mixture allows, for example, the amplification and detection of at least one non-specific nucleic acid sequence, commonly found in all samples (positive control). This makes it possible to verify in particular that the extraction has proceeded well, and that the eluate has reached the reaction zones and that the amplification reaction has proceeded well. Alternatively, the second mixture may be a negative control that does not normally activate unless the cartridge has malfunctioned (e.g., RNA contamination in this control disk). In one embodiment, the test plate may comprise a positive control disc and a negative control disc.

The test plate and the biological test system may be used in particular by an isothermal nucleic acid amplification method called “LAMP” or “RT-LAMP”, but other methods may be used.

A pathogenic species may be a microorganism such as a virus or a bacterium, the nucleic acid sequence of interest thus being specific to the pathogenic species to be detected.

For the “body” of the plate, the term “substrate” or “substrate body” may also be used.

Advantageously, the plate body is of the monolithic type.

With regard to the recovery reservoir, it should be noted that the latter may be provided to collect not only the biological sample, but also the rinse liquid(s) that have rinsed the extraction membrane before its elution, and the remains of the biological sample in the form of eluate after treatment. Typically, the recovery reservoir receives first the sample, then the rinse liquid and finally the eluate from the reaction zones. With the recovery reservoir, no liquid comes out of the plate, only air can come out of the plate. This prevents contamination of the environment and/or the next test.

In various embodiments of the invention, optionally one and/or more of the following provisions, taken alone or in combination, may be further employed.

According to one aspect, the test plate may further comprise at least a first volume of first rinse liquid, and a volume of eluent, contained directly in body housings or contained in pouches arranged in body housings.

The volumes of rinse liquid and eluent are carried on board the plate. The analysis station is thus relatively simple, and only a pneumatic connection is required between the plate and the analysis station. The liquids (rinse and eluent) being on board the plate, they may be different according to the nature of the biological test and the species of nucleic acid of interest which one seeks to reveal, the analysis station remaining the same one. Eventually, the parameters and the software running the station may be updated by downloading but the station hardware remains unchanged.

According to one aspect, the plate is monolithic and contains in a single body the following elements: the extraction membrane, one or two (or more) reaction zones, the volumes of rinse liquid and eluent, the recovery reservoir, and the result reading zone.

According to one aspect, the eluent contained in the eluent volume is RNase-free distilled water and the eluent volume has a volume between 30 microliters and 50 microliters. This water, called “DNase/RNase free water”, elutes the nucleic acid species retained in the extraction membrane and carries them to the reaction zones.

According to one aspect, a compound known as Optigene™ Ibuffer™ (10×Buffer (500 mM Tris-HCl (pH 8.1 @ 25° C.), 300 mM KCl, 300 mM (NH4)2 SO4 and 1% Triton X-100)) may be selected as the eluent.

According to one aspect, the test plate may further comprise a second volume of a second rinse liquid, wherein the first and second volumes of rinse liquid have a volume between 100 microliters and 300 microliters. This volume of a second rinse liquid is used to complete the first rinse before drying. There are thus 3 volumes of liquid on board the plate. Advantageously, no liquid is present in the analysis station. As the liquids (rinse and eluent) are on board the plate, they may be different depending on the nature of the biological test and the target species.

According to one aspect, a single plate edge recovery reservoir may be provided, which recovery reservoir is configured to collect all liquids. Thus not only the remains of the biological sample as eluate after processing, but also the rinse liquid(s) that were used to rinse the extraction membrane prior to its elution are trapped in the single collection reservoir and cannot exit the plate (only air can exit the plate), thus avoiding any contamination of the environment and/or the subsequent test.

According to an alternative aspect, it may be provided that the rinse and elution fluids are not carried on the plate, but are received on the plate from the analysis station. The plate is then very simple, containing no fluids before use. After use, the reservoir contains and traps the liquids used during the test, i.e. the rinse and elution liquids as well as the remains of the initial biological sample.

According to one aspect, the test plate may further comprise at least two valves, preferably of the membrane type, for directing fluid flow from the extraction membrane selectively to the recovery reservoir or to the reaction zone(s).

These valves are used to direct the flow of fluids inside the plate, especially when a reduced number of pneumatic connections between the analysis station and the plate are used. The valves are on board the plate but are controlled from the analysis station.

According to one aspect, the test plate may further comprise an auxiliary reservoir arranged adjacent to the first, and optionally the second, reaction zone(s) so as to be able to supply eluate to the first and second reaction zones by capillary pumping.

This provides a capillary feed to the reaction zones from the auxiliary reservoir, without the eluate flowing through the porous media in the reaction zones and removing the lyophilized reagents from the reaction zones. This also allows for finer control of the volume of eluate that soaks the porous media and hydrates the reaction mixtures, thereby increasing the repeatability of the assay. The porous media may also be made to occupy almost all of the space in the housing to avoid dead volumes that would dilute the reaction mixtures too much. A small air evacuation passage may be provided around the perimeter of the porous media.

According to one aspect, the porous media of each of said first and second reaction mixtures is formed from a paper substrate in the general form of a disc. This utilizes a competitively available material and shape.

According to one aspect, the porous media of each of said first and second reaction mixtures is formed as a paper disk with a radial projection for capillary pumping, the second and third housings therein have a single inlet/outlet port through which the radial projection passes, i.e., the second and third housings are in dead-end configuration. Only the radial projection protruding from the disc is bathed by the eluate flow. The discs are not bathed by the eluate flow, they are supplied with eluate by capillary pumping. This limits the diffusion of freeze-dried reagents from the discs.

According to one aspect, the plate body is formed of a translucent plastic material, such as polymethyl methacrylate (PMMA), polycarbonate (PC), cycloolefin copolymer (COC) or Cyclo Olefin Polymers (COP).

The result may thus be easily read by transparency, as the body of the plate is not an obstacle. It is noted that the optical reading is carried out without moving the plate which remains in place on the base.

According to one aspect, the channels and housings may be formed by machining into the body of the test plate. According to another aspect, the channels and housings may be obtained by molding.

In one aspect, the test plate may further comprise a membrane or vent valve, arranged on at least one of the air outlets, for passing air to the atmosphere and not passing liquid.

A Gore tex™ type cap may advantageously prevent any liquid spillage while allowing air to pass through.

In one aspect, an intermediate air outlet is provided near the inlet of the recovery reservoir to facilitate drying of the membrane after the passage of the rinse liquids.

The drying flow is prevented from flowing through the reservoir over its length.

In addition, it facilitates the advancement of the eluate after elution by expelling air through this port in the single recovery reservoir configuration.

In one aspect, ribs are provided in the recovery reservoir. This provides control of the advance of the liquid front into the recovery reservoir.

According to one aspect, an absorbent pad is provided in the recovery reservoir to capture liquids reaching the reservoir (typically by capillary action) and to prevent leakage of liquid through the air outlet (including when air is injected into the test plate). In particular, the absorbent pad allows to capture amplicons that would reach the recovery reservoir and thus prevent them from escaping from the recovery reservoir to contaminate the plate or the station. An air passage may be provided around the edge of the absorbent pad to allow free flow of air to the air outlet. The recovery reservoir also reduces the force required to move fluids through the cartridge (by capillary action).

According to one aspect, the test plate may have a generally flat parallelepiped shape with two main faces and wherein the air inlet and the inlet for receiving a biological sample to be tested (ET) are arranged on one main face.

This provides simplicity of implementation and design of the analysis station.

According to an optional aspect, the air inlet and the inlet receiving the biological sample to be tested are merged and together form a single fluid inlet.

According to one aspect, all functional interfaces, i.e. inputs and push-button activated elements, are arranged on one main side. This provides simplicity of implementation and design of the analysis station. According to one aspect, all functional interfaces, namely inputs and push-button activated elements, are grouped on one side of the main face (e.g. grouped on three quarters of a length of the face or half). This allows a part of the plate to remain outside the cover of the analysis station, so that the plate may always be identified during processing (the cover will be described later).

According to one aspect, it is provided that all interfaces of the plate with the analysis station are on one side, and the other side of the plate is provided with a rectangular sealing membrane and of uniform thickness. This sealing membrane may be of the ‘PCR adhesive film’ type or of the ‘PSA tape™’ type. This provides simplicity in the construction and design of the analysis station. Different adhesives may be used: one for the discs and another for the rest of the test plate.

According to one aspect, it may be provided that the interfaces of the plate with the analysis station are located on a first side, i.e. a first main side and the inlet for receiving a biological sample to be tested (ET) is arranged on the second side, i.e. the second main side.

According to one aspect, the test plate may further comprise a plate identification element.

This allows the identification element to precisely identify the plate and the test performed, which may be reliably associated with a patient from whom the biological sample to be tested originates. The identification of the plate by the station also allows the test sequence to be adapted to the plate (drying time, heating temperature, heating time, etc.).

According to one aspect, the plate may be configured to receive, by plugging, a collection container containing a biological sample to be tested. This eliminates a risk associated with spilling a biological sample contained in a standard collection tube into the inlet of the insert for receiving a biological sample.

According to one aspect, the test plate may further comprise a non-return valve on the air inlet. In this way, a risk of liquid backflow through the air inlet during the introduction of the biological sample to be tested is avoided.

According to one aspect, the test plate may further comprise a non-return valve at the inlet for receiving the biological sample to be tested. In this way, a risk of liquid backflow through this port is avoided.

These two check valves allow fluid (air or liquid) to flow into the plate and prevent fluid (air or liquid) from flowing out of the plate. Note that in the entire proposed process, nothing can leave the plate except air at the air outlet downstream of the reservoir.

According to one aspect, the first housing, the second housing and the third housing are arranged in close proximity to each other, preferably in a thermal interaction zone with an area of at most 5 cm² or 4 cm². Thus, the extraction membrane and the reaction discs may be heated by a single thermal activation module in the analysis station.

According to one aspect, the test plate comprises two main, opposing faces, and all functional interfaces for the test (i.e. sample inlet, air inlet, rinse volumes, valves, reaction zones) are located on only one of the two main faces.

According to one aspect, there is provided an assembly comprising a test plate as previously described and a rigid support on which the test plate is mounted, the rigid support completely covering the main face not having the functional interfaces. When installed in the station, the rigid plate is positioned between the test plate and the cover. The rigid plate is typically between 0.5 mm and 2 mm thick.

According to one aspect, there is proposed a test equipment comprising a test plate as described above, and a collection container comprising a sealed inlet and a sealed outlet, both arranged at the bottom of the collection container, and a tightly closing lid.

In one aspect, the collection container may be plugged onto the test plate, and the test plate may comprise two needles arranged opposite the sealed inlet and the sealed outlet, respectively, the needles passing through the caps during the plugging process.

According to one aspect, there is provided a biological test system, comprising an analysis station, and one or more test plates as described above, the analysis station comprising at least one pneumatic connector configured to be coupled to the air inlet and/or air outlet.

This allows for a simple connection during the insertion movement of the insert into the test position. A single movement, i.e. lowering the insert into the translational test base, is sufficient to achieve the coupling between station and insert.

According to an aspect, the analysis station comprises at least one of the following features:

a control unit,
an air pump,
one or more actuators (to push on the volumes and/or activate the controlled
valves),
an optical analysis device (to determine the result),
a heating device,
wireless communication means,
an identifier reader,
a hood locking system.

According to one aspect, there is provided an analysis station for a test plate as described above or according to an assembly as described above, the analysis station comprising:

an air pump,
one or more actuators, to push on volumes and/or activate controlled valves,
an optical analysis device for determining results,
a heating device,
a base configured to receive the test plate.

The base may include a cavity shaped complementary to a main face of the test plate, such that the test plate may be positioned by simply snapping into the cavity and removed by exerting a force in the opposite direction to the cavity. The cavity may include an interface zone for the air pump, the one or more actuators, the optical analysis device and the heater, the interface zone being configured to cooperate with a functional interface of the main face of the cartridge.

The optical analysis device and the heating device are, for example, arranged at the level of the cavity, close to each other, preferably in a thermal interaction zone with an area of at most 4 cm².

The analysis station may comprise a cover, rotatably mounted with respect to the baseplate, for example by means of a hinge, which is configured to, in the open position, allow an operator to insert and remove the test plate from the cavity and, in the closed position, to press the plate into the cavity. The cover may be locked by a cover locking system. A Hall sensor may be used to detect the closed position of the cover and allow the lock to be released.

The analysis station may further comprise: a control unit to drive the various components of the station and wireless communication means to communicate the test results to an external device (mobile phone, tablet, computer).

According to one aspect, there is proposed a method of biological testing implemented in a test plate as described above, the method comprising:

S1—passing the biological sample to be tested (ET) in liquid form through the extraction membrane, and direct it downstream to the recovery reservoir,
S2—passing a first rinse liquid through the extraction membrane, and direct it downstream to the recovery reservoir,
S3—passing air through the extraction membrane to dry it,
S4—passing a volume of eluent through the extraction membrane, and directing the eluate that exits downstream to the first, and if applicable the second, reaction zone(s).

Advantageously, the drying of the extraction membrane is carried out in situ by means of an air flow which circulates through the channels and the housing containing the extraction membrane. This ensures that no liquid is left, i.e. that the membrane has been dried out, so that only the eluent provided for this purpose is subsequently carried to the reaction zones, which contributes to the reliability of the test process.

According to one aspect, it may be provided before the drying step S3: S25—passing a second rinse liquid through the extraction membrane, and direct it downstream to the recovery reservoir.

According to one aspect, it may be further provided:

S5—heating at least the first, and if applicable the second, reaction zone(s) to a predefined temperature,
S6—waiting for at least a predetermined time while maintaining the predefined temperature,
S65—waiting for it to cool down or actively cool down by activating a Peltier cell or by means of a convector medium, for example,
S7—illuminating the first and, if applicable, the second reaction zone(s) (31, 32), and receiving a fluorescence level in return,
S8—determining a result.

According to a particular aspect, a step of cooling the reaction zones to room temperature before optical reading is provided.

Advantageously, steps S1 to S8 above are carried out when the plate is in position in the base provided for this purpose, without moving or handling the plate.

According to a particular aspect, a first reference optical reading before heating is provided, which may be performed in order to zero the optical system.

According to one aspect, it may be further provided: S9—assigning the result to a patient file.

As the identification of the plate is bi-univocally linked to the biological sample and its donor, the result is assigned to a patient file bi-univocally to the plate, thus to the biological sample and thus to its donor, i.e. the patient. In this way, a large number of samples can be processed without the risk of misallocation of results.

According to another aspect, there is thus proposed a test plate, for single use, in a biological test system for detecting the presence of one or more pathogenic species (e.g. a nucleic acid sequence of interest), the test plate comprising:

a body in which channels and housings are formed, which are generally isolated from the external environment,
an inlet for receiving a biological sample to be tested (TS) in liquid form, the inlet preferably being sealable or reclosable,
at least one air inlet, and at least one air outlet,
an extraction membrane, arranged in a first housing,
a reaction zone containing a porous media arranged in a second housing and containing a first test reaction mixture in lyophilized form, said reaction mixture allowing the amplification and detection of at least one nucleic acid sequence of interest,
a recovery reservoir,
a result reading zone, comprising at least the reaction zone, the test plate being configured to feed the biological sample to be tested into the extraction membrane, and then, after rinsing, drying and eluting said extraction membrane, to feed the resulting eluate into the reaction zone, in order, after reaction, to deduce a result through the reading zone.

For very large numbers of tests, where the test is already well known, the test plate may be simplified and the control zone dispensed with. Another simpler means of verifying that the eluate has arrived in the reaction zone may be used. For example, a camera in the station may transparently monitor the operations in the plate. For example, a station pressure sensor may be placed in parallel with the air inlet or air outlet. For example, a capacitive sensor in the station may detect fluid displacements. For example, a limit sensor or optical encoder may detect the position of the actuators. For example, a force sensor may detect a force applied by the actuators (via the motor current, for example). This provides a particularly cost-effective solution for the pad with a single reaction disk, the pad being a consumable from the point of view of the test system. All the optional features described above may be applied to this single reaction zone configuration.

Other aspects, purposes and advantages of the invention will become apparent from the following description of several embodiments of the invention, given as non-limiting examples. The invention will also be better understood with reference to the attached drawings in which:

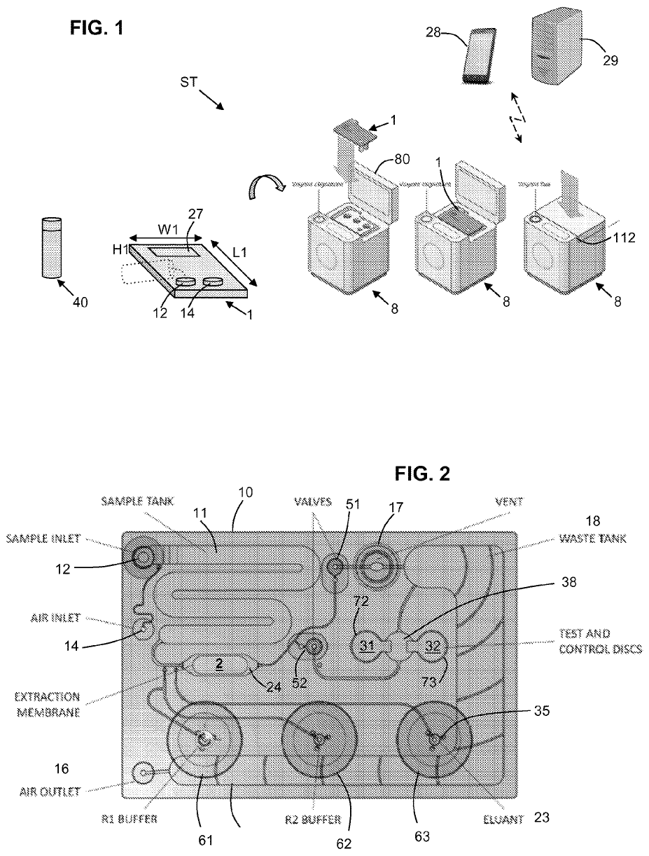

FIG. 1 is a general synoptic view of a biological test system according to the present invention,

FIG. 2 is a schematic view of a first embodiment of a test plate according to the present invention,

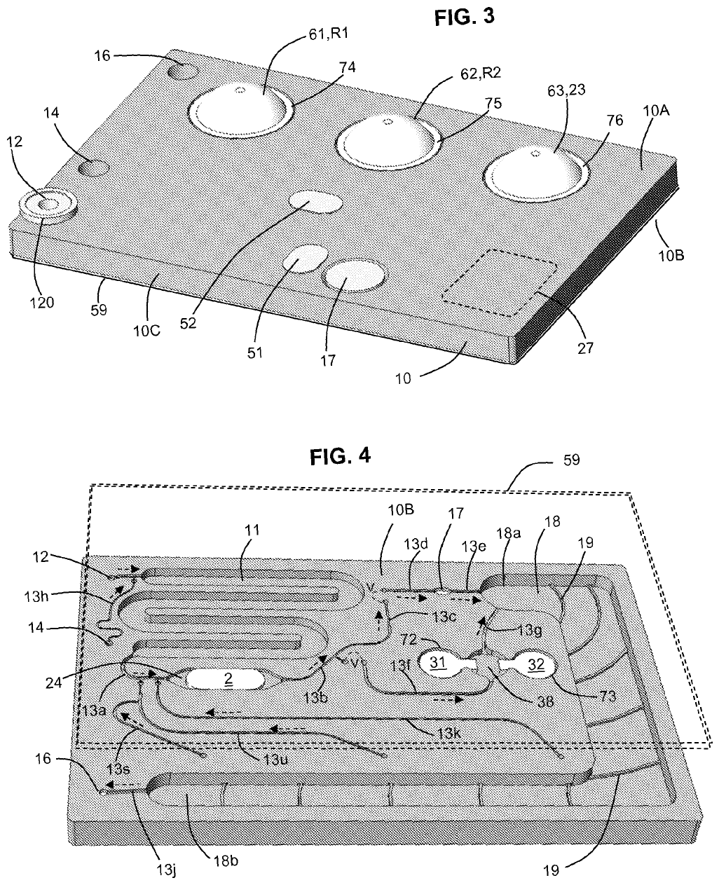

FIG. 3 is a perspective view of the first embodiment of a test plate according to the present invention,

FIG. 4 is a perspective view from the opposite side of the plate in FIG. 2, with the closure membrane separated,

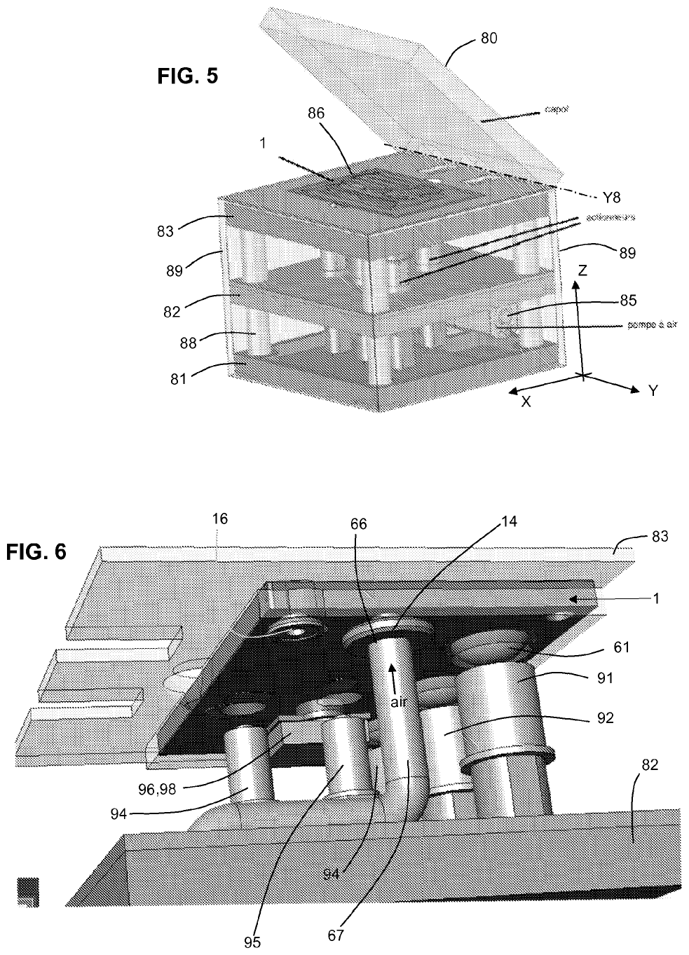

FIG. 5 is a perspective and transparency view of an analysis station compatible with the first embodiment,

FIG. 6 is a partial perspective view from below of the station in FIG. 5,

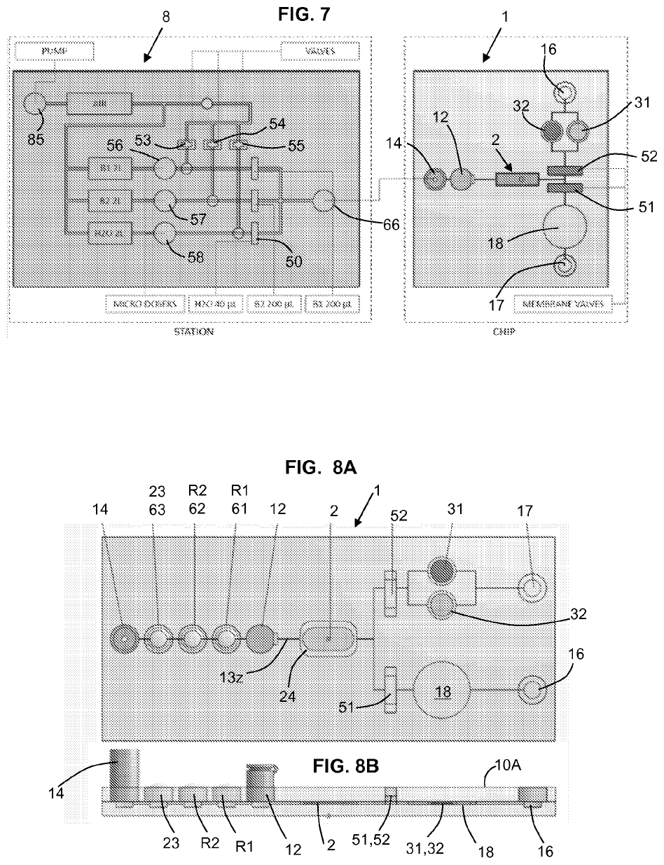

FIG. 7 is a schematic view of a second example of a test plate,

FIGS. 8A and 8B are schematic front and side views, respectively, of a third embodiment of a test plate,

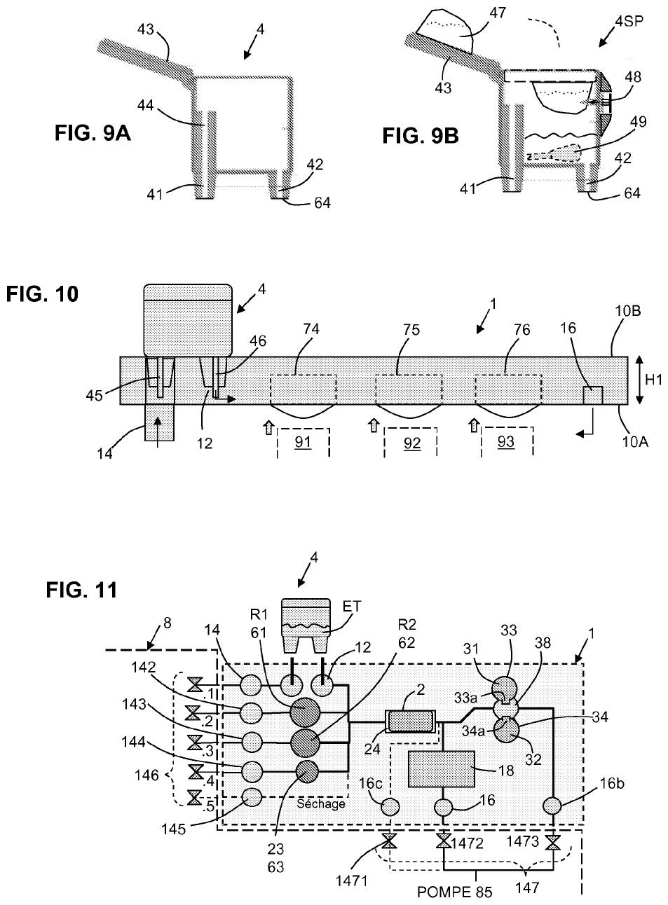

FIGS. 9A and 9B illustrate a collection container according to two particular embodiments,

FIG. 10 illustrates a schematic elevation view of a test plate according to one of the possible examples, in an embodiment adapted to receive the collection container of FIG. 9,

FIG. 11 is a schematic view of a fourth example of a test plate,

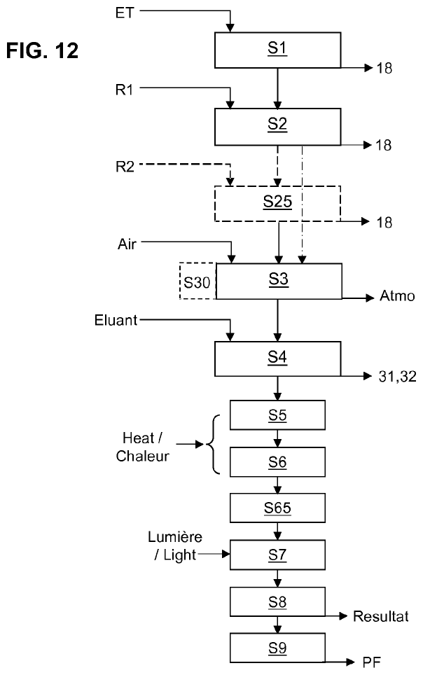

FIG. 12 illustrates the process used in various examples of the plate and/or the analysis station,

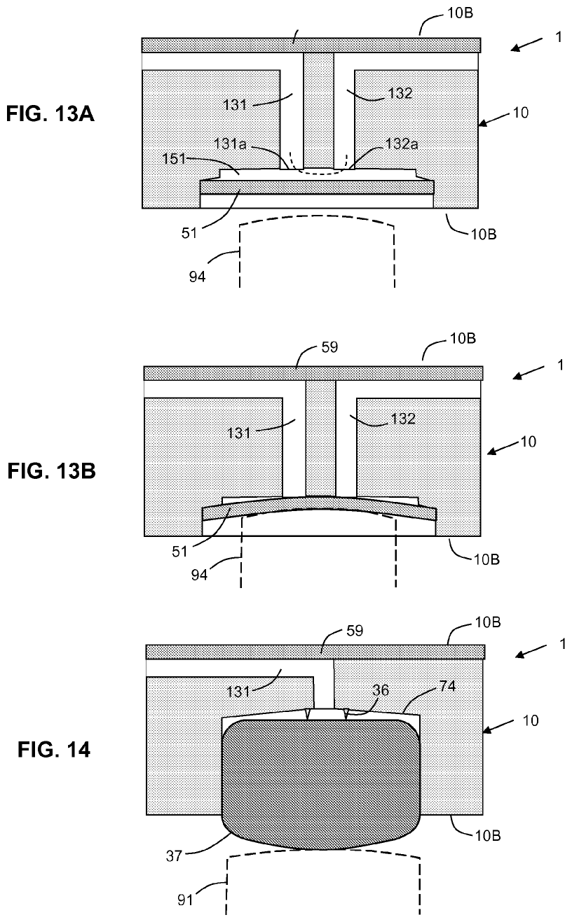

FIGS. 13A and 13B are detail views illustrating a diaphragm type valve,

FIG. 14 is a detail view showing a bag of liquid loaded on the plate and pushed by a piston of the analysis station,

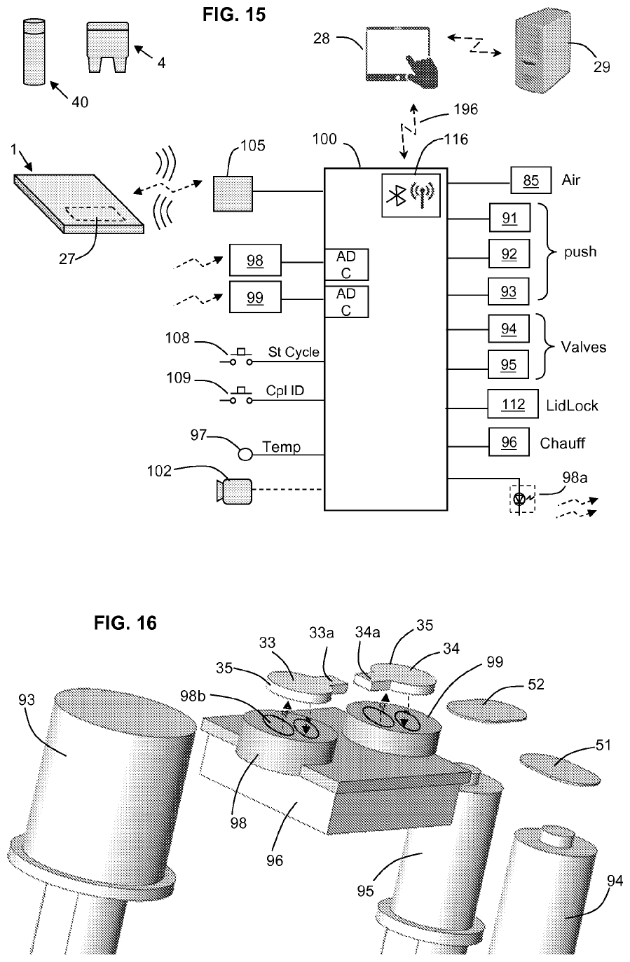

FIG. 15 is a system view centered on a schematic functional illustration of the analysis station,

FIG. 16 is a partial detail view showing the device for optically reading the reaction zones.

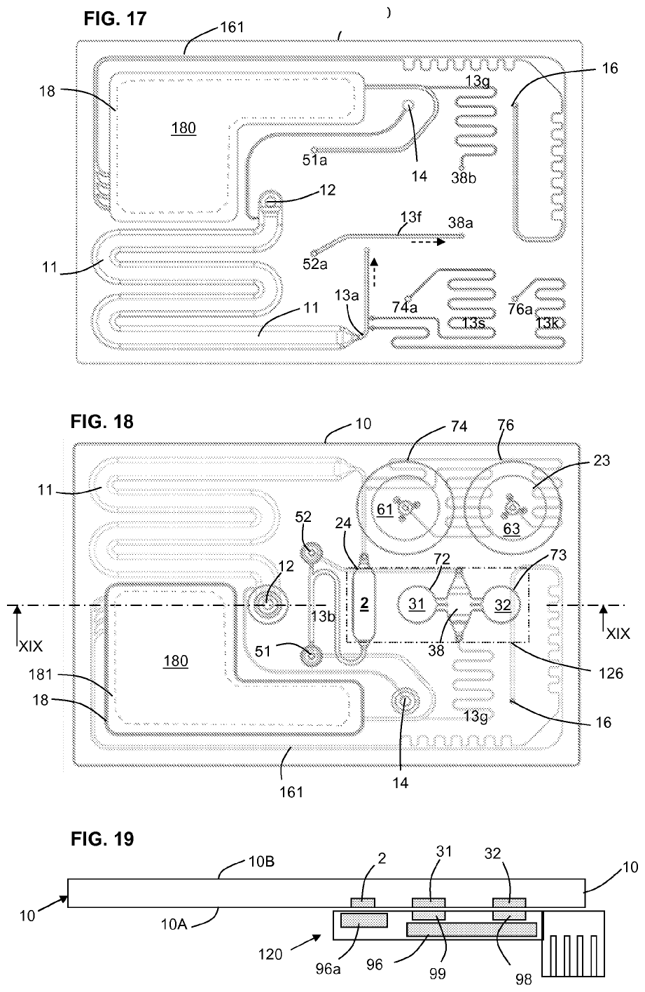

FIGS. 17 to 21 illustrate a fifth example of a test plate embodiment, including:

FIG. 17 is a plan view from the back side of the plate of the upper side in the test position in the station,

FIG. 18 is a plan view from the opposite side of the plate, with the interface and activation elements,

FIG. 19 is a cross-sectional view with the plate and the heating/cooling and analysis device,

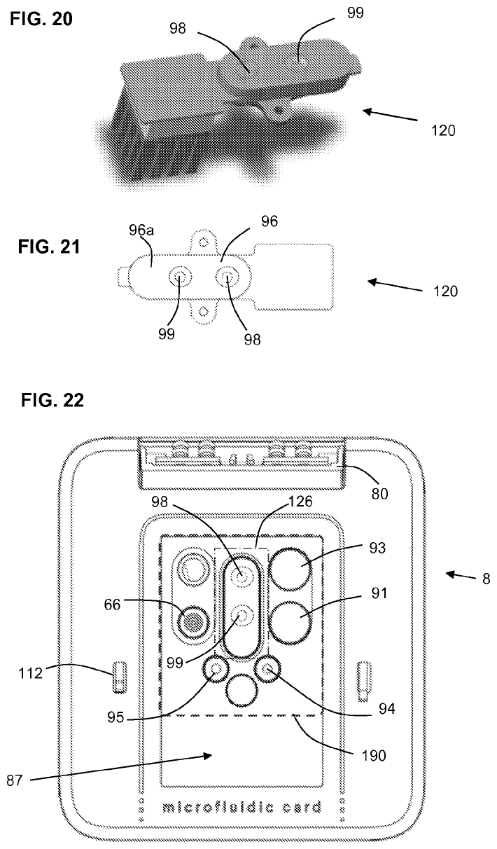

FIG. 20 is a perspective view of the heating/cooling and analysis device,

FIG. 21 is a top view of the heating/cooling and analysis device,

FIG. 22 is a top view of the analysis station with the cover open,

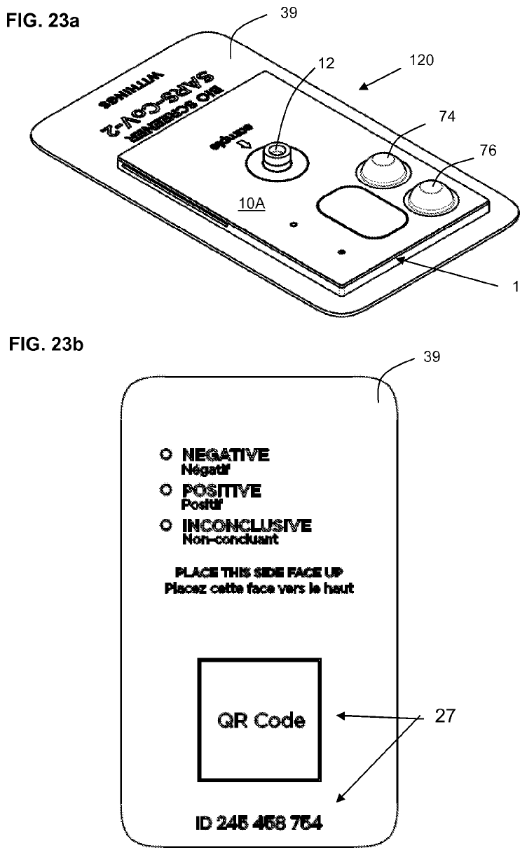

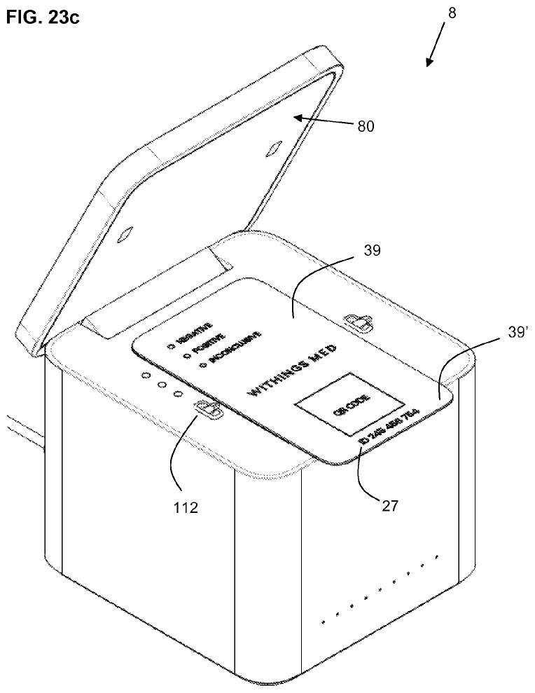

FIGS. 23a, 23b and 23c illustrate two views of the test plate associated with a rigid plate and a view of the test plate with its rigid plate in position in the test station (open cover).

In the various figures, the same references designate identical or similar elements. For reasons of clarity, some dimensions may not be shown to scale.

### System—General

According to the overview shown in FIG. 1, a biological test system ST is provided which comprises an analysis station 8, a test plate 1 and a collection container 40.

The test plate 1 is intended for single use, so there are as many test plates as there are unit tests to be performed. In the example shown here, the collection container 40 is of the standard type, however, we will see later that another, more specific type of collection container may be used.

The collection container 40 is used to collect a biological sample, for example saliva, nasopharyngeal swab, or urine, or even sweat. Although the proposed system is particularly intended for processing human samples, it is not excluded that the proposed system may be used to process samples taken from animals. Optionally, for certain types of tests, blood or lymph may be collected as a biological sample for testing.

Collection of the biological sample in the collection container 40 involves depositing a sample of raw body fluid from the human or animal into a lysis buffer (to disrupt biological cell membranes), then shaking the collection container for a sufficient time of one to several minutes, whereupon a predefined amount of ethanol is poured into the vessel to freeze the sample into a prepared sample state. It is noted that only a small amount of raw body fluid needs to be collected, typically a volume between 0.1 milliliter and 1 milliliter is sufficient for multiple types of testing.

The biological sample in the collection container 40 thus prepared (marked ET) is then poured into an inlet opening of the plate marked 12, whereupon this inlet opening is re-sealed, i.e. the inlet is sealed by a stopper or a sealing membrane.

According to another optional and advantageous configuration, the inlet port 12 of the plate is provided with a non-return valve. This valve allows entry into the insert but prevents reverse movement. The valve may be mounted on the inlet port by a retaining ring, for example. The valve may be a silicone valve, such as a duckbill valve, a dome valve, an umbrella valve, etc.

The plate 1, which now contains the biological sample to be processed (also referred as ‘to be tested’, hence the name ‘test plate’), is placed on a test platform in the analysis station 8, which then has its cover 80 open. Then the door (the cover) is closed and a user initiates a processing cycle, i.e. a test cycle. At the end of this cycle, a test result is given by the analysis station, in a form which will be described later. The test plate and the analysis station may have various functions and features; several example embodiments are shown below.

The result is obtained in a few tens of minutes. Most often the result is obtained in less than 1 hour. For many types of tests, the result is obtained in less than 50 minutes all inclusive.

Such a system may be used in the context of an analytical laboratory, but due to its simplicity and speed of implementation, this biological test system may be used beyond that, in a very broad context, i.e. in a pharmacy, in a medical practice, in a general care facility, in a quarantine and quarantine release system. This biological test system may also be used in a veterinary vehicle for testing animals.

### Brochure—First Example

FIGS. 2 to 6 illustrate a first example of embodiment of a test plate 1 and an analysis station 8 compatible with its processing.

The present test plate comprises a body 10, which may also be referred to as a substrate. Channels and housings as seen below are formed in the plate body.

Generally speaking, the term ‘plate’ must be taken here in a very broad sense, the plate is here a support which may take any geometrical form. By abuse of language it may also be called ‘chip’ or ‘puce’, a term which comes from the logic ‘Lab-on-a-chip’. Instead of test plate, we could also use ‘cartridge’ or ‘cassette’.

All the processing operations performed on the biological sample to be tested are carried out in this plate, in particular the extraction of the DNA/RNA molecules, rinsing and elution, the plate also comprising at least one result reading zone.

In the illustrated example, the test plate has a generally flat parallelepiped shape, with two main faces, namely a first main face 10A called the interaction face and a second main face 10B called the rear face. In addition, a peripheral slice 10C forming the other four faces extends between the two main faces. In the illustrated example, the thickness of the plate noted H1 is between 3 mm and 20 mm. The length L1 is between 20 mm and 80 mm and the width W1 is between 10 mm and 60 mm. The geometrical configuration may be similar to a credit card format, but with a slightly greater thickness.

The plate is identified by an identification element 27 of the plate, which may be arranged on the plate (i.e. QR Code) or in the plate as in the case of an RFID, NFC chip or the like. The identification element may be even simpler, such as color coding or mechanical coding. The identification element 27, when digital, allows for unambiguous unitary identification of the test plate, and incidentally for storage of the result in the identification element 27 at the end of the test prior to opening the door/cover 80.

Furthermore, the biological sample is associated with an individual from whom the biological sample originates, which allows for a bi-univocal association between the biological sample, the plate and the donor.

The RFID or NFC chip may also identify that a plate is mounted in the station. This may condition the activation of the station lock.

In the illustrated example, the plate is obtained from a homogeneous parallelepipedic block of material by removal. Milling and drilling are carried out from the rear side 10B to obtain the above-mentioned channels and housings.

The plate body is formed from a translucent plastic material, such as polymethyl methacrylate (PMMA), polycarbonate (PC), cycloolefin copolymer (COC) or Cyclo Olefin Polymers (COP).

Instead of starting from a homogeneous parallelepipedic block, we may start from a moulded form where the main housings are already formed by moulding and only small diameter drillings remain to be made.

It is also possible to obtain the insert with all its channels and housings directly from the mould, without reworking.

In addition, instead of a material removal method, an additive manufacturing process such as 3D printing may be used.

It is noted that the chosen substrate forms an effective barrier to contain the fluids under consideration, it does not allow the passage or absorption of air and rinse liquids discussed below.

### Canals, Reservoirs and Housings

On the rear face 10B, to close the channels and the housings, a closing membrane 59, for example a bio-compatible adhesive plastic film, is glued. Alternatively, a rear cover may be welded, for example by ultrasonic welding. A rigid plate, as described below, may be used as a back cover. Said rigid plate may have larger dimensions than the plate itself.

Thus, channels and housings are generally isolated from the external environment. The channels are generally marked with the reference 13. Fluid flows through these channels, which may be liquid or air. The channels have a small cross-section compared to the dimensions of the housings. Among the housings, there is a so-called upstream reservoir noted 11 which forms a reservoir for the temporary storage of the biological sample to be tested ET; this space is a coil in the illustrated example. But it could have another shape. There is also a downstream reservoir, also called a recovery reservoir 18, which is downstream of the channels carrying liquid fluids, and which is sized to hold: the volume of biological sample to be tested input+the volumes of rinse liquid+the volume of eluate. The volume of this recovery reservoir 18 is between 0.1 milliliter and 2 milliliters. The recovery reservoir 18 extends from an inlet end 18a to a bottom 18b.

According to an optional configuration, the recovery reservoir 18 contains an absorbent pad 180. This absorbent pad has a blotting effect and captures liquids. It is intended that the absorbent pad 180 does not occupy the entire available volume in the recovery reservoir 18, and to leave at least one air passage 181 between the pad 180 and the walls of the reservoir so that air may escape through the outlet 16. An outlet channel 160 may connect the air passage 181 to the air outlet 16.

Returning to the channels, and with particular reference to FIG. 4, a channel 13h connects the air inlet 14 to the upstream reservoir space 11.

A further channel 13a connects the upstream reservoir space 11 to a first housing 24 containing an element known as the extraction membrane which will be seen later.

A further channel 13b connects the housing 24 to a bypass valve 52. From there, a further channel 13f connects the valve outlet to the area containing the reactants, in particular to an auxiliary reservoir 38.

A further channel 13c extending the channel 13b connects the bypass valve inlet to a further valve 51 which allows or stops the passage of fluid downstream to the recovery reservoir 18, terminating at an air outlet 17. A further channel 13e extends the channel 13d and opens into the recovery reservoir 18.

Channels 13s, 13u, 13k respectively connect the housings marked 74, 75, 76 to the channel 13a upstream of the housing 24 of the extraction membrane.

A channel 13f connects the outlet of the bypass valve 52 to an auxiliary reservoir 38 interposed between the housings 72, 73.

In addition, a channel 13g connects the auxiliary tank 38 and opens in the vicinity of the inlet end 18a of the recovery reservoir 18.

Finally, a channel 13j communicates the bottom portion 18b of the recovery reservoir 18 with the atmosphere via an air outlet 16.

With respect to the channels 13, small dotted arrows illustrate in FIG. 4 the direction of flow of the fluid (liquid and/or air) in these channels. The terms ‘upstream’ and ‘downstream’ refer to the flow directions indicated. Generally, there is no two-way flow in the channels 13.

As already mentioned, an inlet 12 is provided for receiving the biological sample to be tested ET in liquid form. This inlet is sealable or reclosable with a stopper. Alternatively, this inlet is equipped with a non-return valve. Thus, after the biological sample has been poured in and closed, the plate may be turned over without any risk of spilling any of the biological sample.

Once poured into the plate, the biological sample liquid is contained in an inlet reservoir 11, which in the illustrated example is in the form of a coil-shaped housing.

In addition, the plate includes an air inlet marked 14. This allows air to be supplied to the plate, particularly from the analysis station.

In an embodiment not shown in the figures, the air inlet 14 and the inlet 12 receiving the biological sample to be tested ET are merged and together form a single fluid inlet.

### Extraction Membrane, Process Fluids and Valves

In addition, the plate comprises an extraction membrane marked 2. The extraction membrane may be a silica membrane or a cellulose membrane. The extraction membrane has a porous structure, i.e. a honeycomb structure. The function of the extraction membrane is the isolation of DNA/RNA molecules and the removal of proteins and other short-molecular waste products as will be described below.

In one example, the silica matrix of the extraction membrane is able to selectively bind DNA/RNA under high ionic strength and pH ≥7 conditions. Impurities are removed by rinses and after drying, the DNA molecules and RNA sequences are eluted in a solution in low ionic strength and pH ≥7. The silica matrix may be in a format of glass powder, glass/silica particles or glass fibers. In particular, rinsing also removes residual lysis buffer and drying removes residual ethanol. The eluent must not be contaminated by lysis buffer or rinse liquid, which may otherwise inhibit the amplification reaction.

For more details on the extraction membranes used herein, reference may be made to EP1463744.

It is noted that the extraction membrane 2 occupies the entire housing 24 in which it is arranged in the direction transverse to the flow of fluids that are passed through the extraction membrane 2. In other words, the flow of fluids (rinsing, drying, elution) cannot bypass the extraction membrane 2.

According to one example, the GF/F Whatman membrane of source Sigma-Aldrich™ may be selected for the extraction membrane. According to another example, the GF/D Whatman membrane from source Sigma-Aldrich™, which has larger pores than the GF/F Whatman membrane and allows for accelerated flow, may be selected for the extraction membrane.

For example, the length of the membrane may be in the range of 10 mm, the width of the membrane may be in the range of 3 mm to 5 mm and the thickness of the membrane may be in the range of 0.3 mm to 0.6 mm.

In addition, the plate includes processing fluids that are predisposed on the plate, i.e. present on the plate itself. They may be said to be on board or on the plate.

In the illustrated example, there is provided a first volume 61 of rinse liquid R1 arranged in housing 74, a second volume 62 of a second rinse liquid R2 arranged in housing 75, and a volume 63 of eluent 23 arranged in housing 76. These volumes/products are contained directly in housings of the body 10 or contained in pouches 37 which are arranged in housings of the body. In the bag/blister configuration, these bags are prepared in advance and have a specific capacity. The liquid of interest is contained in this sachet, for example of aluminium foil, and there is no direct contact between the body of the plate and the liquid of interest before actual use by piercing/tearing the sachet.

The contents of these fluid volumes may be discharged and directed to the extraction membrane 2 through the aforementioned channels 13s, 13u, 13k as will be seen later, in response to activation.

It is noted that the presence of the second rinse liquid R2 is optional. Indeed, depending on the type of test, the housing 75 may remain unused or be absent, only one rinse liquid is used.

In the illustrated example, the first and second rinse fluid volumes R1, R2 have a volume between 100 microliters and 300 microliters. Of course, the invention also works for smaller rinse volumes and also for larger volumes.

Regarding the volume of eluent, it may be between 30 microliters and 50 microliters. Here again, the invention works also for smaller eluent volumes and also for larger eluent volumes.

The eluent is preferably a special water, i.e. RNase and Dnase free distilled water, so-called “Dnase/Rnase free water”. In another example, a Tris/EDTA buffer is used. For the eluent, a compound known as Optigene™ Ibuffer™ may also be chosen as the eluent. In addition, the eluent may also contain salts useful for the reaction.

The eluent has a low ionic strength and has a pH ≥7.

For more details on the type of eluent used here, reference may be made to EP1463744.

In addition, in the illustrated example, the plate includes two routing valves that serve to direct or stop a flow of fluid through the channels 13.

Specifically, the two valves allow a flow of fluid from the extraction membrane 2 to be directed selectively to the recovery reservoir 18 or to the reaction zones.

A first valve 51 may selectively close access to the recovery reservoir 18. Upstream of the first valve 51 is channel 13c and downstream is channel 13d (FIG. 4).

A second valve 52 can selectively open access to the reaction zones. Upstream of the second valve 52 is the channel 13b and downstream is the channel 13f (FIG. 4).

The valves 51, 52 are in the illustrated example membrane type valves. A deformable membrane selectively opens or closes a passage between two neighbouring orifices, the membrane being placed in a closed housing, the two orifices being the only passageways available for the fluid.

FIGS. 13A and 13B illustrate a deformable membrane 51, which selectively opens or closes a passage between two channels 131, 132.

The deformable membrane bounds a chamber 151 from below. The body of the plate delimits the chamber 151 on the sides and on the top, except where the channels open, with a first port 131a (first mouth) and a second port 132a (second mouth). In FIG. 13A the deformable membrane 51 is at rest (flat shape at rest) and the passage between the two mouths 131a, 132a is open (dotted arrow circulation). In FIG. 13B under the effect of a mechanical action of pushing by the element noted 94, the deformable membrane 51 is flexed and comes to bear on the ports (two mouths) 131a, 132a; the passage is between the two mouths 131a, 132a is then closed.

The diaphragm valve has been shown in a normally open configuration, but a normally closed diaphragm configuration may also be used.

The air outlet 16 is arranged at the bottom of or in communication with the bottom of the recovery reservoir, while the intermediate air outlet 17 is arranged near the inlet of the recovery reservoir. Either may be fitted with a Goretex™ or equivalent cap which prevents any liquid spillage while allowing air to pass through.

For the air outlet 16 of the reservoir bottom, depending on the configuration of the reservoir and in particular if it has an excess volume compared to the storage requirement, the Goretex™ cap is optional.

It is noted that there could be only the air outlet 16 of the reservoir bottom but to facilitate the drying of the membrane after the passage of the rinse liquids, and the advancement of the eluate through the channel 13g the intermediate air outlet 17 is arranged which it must be equipped with a Goretex™ cap or equivalent.

### Reaction and Reading Zones

Further, the plate comprises a first reaction zone 31 containing a porous media 35 containing a first test reaction mixture 33, and a second reaction zone 32 containing a porous media 35 containing a second control reaction mixture 34.

The first and second reaction zones are arranged in the second housing 72 and the third housing 73 respectively.

In the illustrated example, an auxiliary reservoir 38 is provided adjacent to the first and second housings 72, 73 for indirectly feeding the porous media. Thus, the porous media in the reaction zones 31, 32, i.e. the disks of the first and second reaction mixtures 33, 34 are supplied with eluate by the inflow of eluate into the auxiliary reservoir 38, without the eluate passing through the disks.

According to an advantageous optional arrangement, in each reaction mixture disc 33, 34, a radial projection 33a, 33b is provided for the capillary pumping function. Only the radial projection 33a, 33b is bathed directly by the eluate flow. The disks are not bathed by the eluate flow, they are supplied with eluate by the capillary pumping. It is noted that the second and third housings 72, 73 which enclose the disks have a single inlet/outlet port through which the radial projection passes, in other words said housings are in a dead-end configuration. It is noted that the contact interfaces between the disks and the reservoir are minimized, as are the dead volumes around the disks so as to limit the diffusion of the reagents contained on the disks.

Both test and control discs may be selected from the Cytiva™ (formerly GE Healthcare™ Life Sciences™) source GF/DVA Whatman. For example, the discs have a diameter between 5 mm and 6 mm and a thickness between 0.7 mm and 1 mm.

According to one example, each disc occupies the entire housing in which it is placed; there is no clearance or space available for eluent to lodge. In one embodiment, it is provided that the air initially contained in the disc may escape during rehydration of the disc. For example, a groove is provided under the discs and a leakage passage to facilitate this air escape.

In one example, the integrated reagents are H2O, dNTPs, 10×isothermal buffer, MgSO4, betaine, intercalating agent, primer mix, Bst 2.0 W S enzyme, and AMV-RT (enzyme polymerase and reverse transcriptase) enzyme. In one example, this may be the WarmStart LAMP Kit from New England Biolabs™.

The test reaction mixture contained in the porous media thus comprises, in one example, the reverse transcriptase which allows for the transcription of RNA into DNA, the set of primers specific to the DNA of the pathogen, the DNA polymerase an isothermal buffer containing deoxynucleotide triphosphates (dNTPs), which are necessary for DNA amplification, MgSO4, which acts as a cofactor and catalyst for the reaction, and betaine, an additive often used to improve the amplification of DNA sequences. Finally, a fluorophore or colorimetric probe is included to reveal the amplification reaction when it occurs.

The first and second reaction mixtures 33, 34 are in freeze-dried form. This form is stable and allows for long storage of fresh plates before use.

The first and second reaction mixtures 33, 34 are in a general disc form.

According to an advantageous optional arrangement, the porous media 35 is made of paper. Its capacity to absorb water free of RNAase and/or DNAase (eluent) is known, controlled and repeatable, which contributes to the reliability of the test.

The result is read by an optical measurement, in particular a first optical device 98 arranged in the analysis station 8 opposite the first reaction zone 31 and a second optical device 99 arranged in the analysis station 8 opposite the second reaction zone 32. An illumination element 98a, which may or may not be common, and an imaging device, an optical sensor or photodiodes are provided in the analysis station 8 for receiving radiation reflected (or transmitted) by the porous media of the first and second reaction mixtures 33, 34.

In the example shown, the fluorescence properties of the porous media where the nucleic acids have multiplied are used.

Said first and second reaction mixtures allowing the amplification and detection of one or more nucleic acid sequences.

It is noted that a result reading zone is provided, comprising at least the first and second reaction zone 31, 32. In the case where the body is formed from a translucent material, the result reading zone may be considered to be as wide as the plate itself. If the substrate used for the body is not transparent or not sufficiently translucent, a specific transparent window for reading the result may be provided.

The fluorescence response of the first and second reaction zones is determined to derive a test result.

Specifically, the fluorescence response of each disc is compared before and after heating. If the fluorescence response of the control disk is low, then the test is declared null.

If the fluorescence response of the control disc is high and the fluorescence response of the test disc is low, then the test is declared negative.

If the fluorescence response of the control disc is elevated and the fluorescence response of the test disc is also elevated, then the test is declared positive.

The first test zone amplifies DNA specific to the pathogen of interest. The second zone (control disk) consists in amplifying the non-specific RNA from the sample. In fact, the two reaction mixtures are identical except for the set of primers used. In the case of the control, the primers used are non-specific to the virus (pathogen of interest) but cooperate with an RNA sequence still present in the incident sample/eluate. The purpose of the control is to confirm that both the extraction step and the amplification step have been successful, thus eliminating the option of a false negative.

It is not excluded to deliver a richer result than a binary result. Provided that the fluorescence response of the control disc is high, the fluorescence response intensity of the test disc may be translated into an index ranging from 0 to 1.

It should be noted that instead of a fluorescence response measurement, other optical methods may also be used, such as a colorimetric method, a photometric method, a transparency coefficient method.

It should also be noted that reaction mixtures may be optically analyzed after reactions, either by reflectivity measurement or by transmissivity measurement (transmitter and receiver on both sides of the plate).

It should also be noted that a reference optical reading is taken before heating, to allow the optical system to be “zeroed” at ambient radiation conditions and with the plate as it is in transparency. After reaction heating and cooling, the new optical measurements are taken with the reference measurement before heating (differential method).

### Analysis Station

The analysis station 8 comprises a frame 89 comprising structural columns 88. The analysis station 8 is in the form of a parallelepipedic box. In one example, this box is close to a cubic shape. In one example, the side of this cubic shape has a length between 25 cm and 40 cm. However, the analysis station could have any shape. The maximum width, length or depth of the analysis station 8 is less than 40 cm, or less than 20 cm, or even less than 12 cm (between 11 cm and 12 cm).

In the illustrated example, the analysis station 8 comprises a bottom stage 81, an intermediate stage 82 and a top stage 83.

The analysis station 8 includes a base 86 configured to receive a test plate 1 and subject it to a series of operations. The base 86 is arranged in the upper stage 83 and includes guides for positioning the plate precisely in the desired location for mechanical, pneumatic, thermal and optical interface between the plate and the station to occur.

For example, the base 86 includes a cavity 87 having a shape complementary to the main face of the cartridge which includes the functional interfaces. The cartridge may thus easily fit into the cavity 87, thus ensuring easy and correct positioning of the cartridge in the station.

The analysis station 8 comprises a cover 80 mounted on the frame by means of an articulation, for example by means of a pivoting movement of axis Y8 (hinge type).

A cover locking system 112 is provided. Indeed, when test operations are in progress on a test plate, the cover is closed and locked. When the test is complete and the result is obtained, the locking system unlocks the cover. An operator may then open the cover, remove the test plate and place a new test plate on the baseplate to be processed.

The cover 80 typically does not include a functional interface with respect to the fluidic or pneumatic circuitry. In other words, the cover does not house any component configured to interact with the test plate 1 during a test. This simplifies the design of the test station 8.

A Hall effect sensor is used to detect the state of closure of the cover. The sensor data is used to activate the lock. Thus, an NFC/RFID signal indicating that the test tag is in place and a signal from the Hall sensor indicating that the cover is closed are required to trigger the cover lock.

The analysis station 8 includes an air pump 85. This may be a booster pump or a vacuum pump, depending on the plate/station configuration.

The analysis station 8 includes a heating device 96. The heating device may be supplemented by a cooling device, such as a fan, a heat sink (e.g., finned heat exchanger, the heat sink being positioned above the fan, for example) and/or a Peltier cell (the same as for heating, for example), to force cooling. The analysis station 8 includes a temperature sensor 97 for regulating the control of the heating device. The heating device may be a resistive heater, a Peltier cell, or may include one or more bodies, typically blackbody(s), disposed proximate to the reaction zones and infrared illumination to heat said blackbody(s). The station controls the heating device to have a plateau at a certain temperature and over a certain time period. For example, the plateau temperature is between 50° C. and 70° C. In one example, the plateau temperature is 65° C. The duration of the plateau is between 20 minutes and 40 minutes. The analysis station may be operated with a predetermined dwell time (parameter). In an alternative mode, the end of the dwell time may depend on the optical analysis of the reaction zones, e.g. the result may be read before the predefined time, or in the opposite case, the optical analysis of the reaction zones may identify an error.

The analysis station 8 comprises a control unit 100, the synoptic of which is shown in FIG. 15.

The analysis station 8 comprises an optical analysis device 98, 99 for determining the result, as already mentioned above.

The analysis station 8 comprises wireless communication means 116. These means allow the result to be transmitted to a remote entity.

The analysis station 8 may include, on the intermediate board 82, one or more actuators 91-95 for pushing on the volumes and/or activating the controlled valves, according to the various example embodiments. Specifically, a first actuator 91 provides a mechanical action on the volume of the first flush volume 61. A second actuator 92 allows for mechanical action on the volume of the second flush volume 62. A third actuator 93 provides mechanical action on the eluent volume 63. A fourth actuator 94 provides mechanical action on the first valve 51. A fifth actuator 95 provides mechanical action on the second valve 52. The actuators may be referred to as ‘pushers’.

The analysis station 8 includes at least one pneumatic connector 66 configured to be coupled to the air inlet 14 of the plate. Pneumatic tubing 67 is provided to connect the air pump 85 to the pneumatic connector 66.

In other configurations, the pneumatic connector(s) may be connected to different air inlets or outlets.

The analysis station 8 includes a user interface in the form of a control button 108, 109 and/or a touch screen.

The analysis station 8 may comprise a device for reading the identification element 27 of the plate(s) 105.

The analysis station 8 may include a camera 102. This camera may be used to check the movement of liquids in the plate.

With reference to FIG. 14, the discharge of a rinse fluid contained in a pouch 37 was illustrated. The top end of the actuator 94 pushes on the pouch/bag. Spikes 36 are provided in the bottom of the housing 74. When the pouch 37 is squeezed, the pins 36 are pressed into the pouch and tear the pouch, releasing the liquid contained therein into the channel 131.

With reference to FIG. 16, the optical analysis device and its arrangement with respect to the disks 35 has been illustrated in more detail.

An LED 98a illuminates one disk 35 and the radiation received in return is captured by one or more photodiodes 98. On the other disc, the same process takes place with one or more photodiodes 99.

### Rigid Plate Support

With reference to FIGS. 23a-c, the test plate 1 may be mounted on a rigid support 39 which may protrude from the station when the test plate is installed thereon. In one embodiment, the rigid support is a plate (e.g., parallelepipedic) with dimensions in width and length greater than that of the test plate but with a dimension in thickness less than that of the test plate. This means that one 10B of the main faces 10A, 10B of the test plate (i.e., the main face not having a functional interface) may be entirely included on the rigid support. Such a rigid support 39 allows easy handling of the plate 1 without touching the main face 10 with the functional interface, allows easy removal of the test plate 1 from the station 8 (simply grasp the rigid part and pull away from the cavity 87), and provides, on the side of the rigid support opposite to the test plate 1, a free surface on which information, such as the identification element 27 of the test plate 1, may be displayed.

In particular, there is no need for an automated plate ejection and insertion system, thus eliminating the need for a motor and other mechanical/electrical components. This improves the compactness of the station 8.

The test plate 1 may be glued or welded to the rigid support 39.

When the test plate 1 is placed in the station 8 and the cover 80 is closed, one end 39′ of the rigid support may remain accessible (“visible portion”) to a user. On the visible end may be offset the identification element 27 (or a part thereof) of the test plate (in particular when it is an alphanumeric reference, a bar code, a QR code or other elements requiring to be visible to an operator), which is thus visible by the operator even when the cartridge is installed in the station. This allows for a simple, fast and non-invasive verification of the cartridge identification.

The thickness of the rigid support is, for example, between 0.5 mm and 3 mm, or 1 mm and 2 mm.

The cover 80, when closed about the axis Y8 comes into contact with the rigid support 39 to press the test plate 1 into the cavity 87 and thus ensure the functionality of the interface between the test plate and the interface zone 90 of the station 8.

By rigid, it is meant sufficiently rigid to be able to handle the plate; but the rigid support may bend slightly (credit card type).

### Process

In FIG. 12, the steps of the process have been illustrated. In the first example where a standard collection container 40 is used, a for example saliva was collected, lysed and then inhibited with ethanol. This resulted in a ready to biological sample to be tested, which was poured into the inlet 12 and is located in the upstream reservoir 11.

The First Step of Interest is to:

S1—passing the biological sample to be tested ET in liquid form through the extraction membrane 2. In practice, the biological sample ET is pushed through the extraction membrane 2 from the upstream reservoir 11 to the recovery reservoir 18 by means of an overpressure of air. The first valve 51 is open while the second valve 52 is closed.
Nucleic acids and other molecular species are retained by the extraction membrane 2.
It is noted that the air outlet 16 allows air in the recovery reservoir to escape as the recovery reservoir fills with liquid.
Then we will proceed to the rinsing, in a step noted S2.
S2—passing a first rinse liquid R1 through the extraction membrane 2, and directing it downstream to the recovery reservoir 18.
Optionally, a second rinsing pass may be used, by means of step:
S25—passing a second rinse liquid R2 through the extraction membrane 2, and directing it downstream to the recovery reservoir.
Then we will proceed to drying, in a step noted S3.
S3—passing air through the extraction membrane 2, to dry it. The station sends air under pressure into the air inlet 14 which runs through channels 13h, 11, 13a, 13b, 13c, 13e, 13j. The air can exit at the bottom of the reservoir through the outlet 16 but also the air can exit through the intermediate air outlet 17. Step S3 may also include: heating the membrane, to accelerate drying. This heating step may be alternative or complementary to the step of passing air through the extraction membrane to dry it. The extraction membrane is then eluted, in a step noted as S4.
S4—passing a volume of eluent through the extraction membrane 2, and directing the eluate exiting downstream to the first and second reaction zones 31, 32. The first valve 51 is closed while the second valve 52 is open. Other steps follow:
S45—optionally, a reference optical reading is taken before heating, to allow the optical system to be “zeroed” under ambient radiation conditions and with the plate as it is in transparency,
S5—heating at least the first and second reaction zones 31, 32 to a predefined temperature,
S6—waiting for at least a predetermined time while maintaining the predefined temperature,
S65—allowing to cool or ventilate to cool to room temperature,
S7—illuminating the first and second reaction zones 31, 32, and receiving a fluorescence level in return,
S8—determining a result.
Finally, the method includes assigning the result (step S9) to a patient file PF.

### Second Example of a Realization

FIG. 7 illustrates a second example of an embodiment. For the elements not commented on in the following paragraphs, it should be considered that these elements are identical or similar to what has been described for the first example embodiment.

In the second embodiment, the volumes are not on board, i.e. the rinse fluids R1, R2 and elution fluids 23 are not on board the plate, but are received on the plate from the analysis station 8. The plate is then very simple, it does not contain any liquid before use. After use, the recovery reservoir 18 contains and traps the liquids used during the test, i.e. the rinse and elution liquids as well as the remains of the initial biological sample.

The analysis station 8 then comprises micro-dosers, respectively 56, 57, 58 for the three fluids R1, R2, eluent 23, the result of the 3 micro-dosings is then found in three intermediate buffers 50. The analysis station 8 then includes selection valves, respectively 53, 54, 55 to supply air at overpressure to the channels of the three fluids. The three channels meet downstream in a single outlet channel. There is a single pneumatic interface 66 with the plate, at the air inlet 14.

### Third Example of a Realization

FIGS. 8A and 8B illustrate a third example of an embodiment. For the elements not commented on in the following paragraphs, it should be considered that these elements are identical or similar to what has been described for the first example embodiment.

The internal channels of the plate have been simplified. A common 13z channel trunk is connected successively from upstream to downstream to the following from upstream to downstream:
the air inlet 14,
the volume of eluent 23,
the volume of the second rinse liquid R2,
the volume of the first rinse liquid R1,
the inlet 12 of the biological sample,
the extraction membrane 2 in its housing 24.

Downstream, there are two diverging branches, each opened or closed by the first and second valves 51, 52 respectively.

For rinsing and drying the first valve 51 is opened and the second valve 52 is closed.

To supply the eluate to the first and second reaction zones, the second valve 52 is opened and the first valve 51 is closed.

In this example, the eluent stream passes through the reaction mixture media 33, 34. But one could also have an auxiliary reservoir as previously described.

### Fourth Example of a Realization

FIGS. 9 to 11 illustrate a fourth embodiment. For the elements not commented on in the following paragraphs, it should be considered that these elements are identical or similar to what has been described for the first example embodiment.

According to the present invention, in its fourth illustrated embodiment, the collection container is not standard. The collection container 4 comprises an inlet 41 and an outlet 42, both arranged at the bottom of the collection container, and a tightly closing lid 43.

Each of these inlets and outlets 41, 42 is sealed when new, i.e. a cover 64 hermetically seals each of these inlets and outlets.

A chimney 44 is provided, forming a channel inside the container and supplying air which will arrive through the inlet 41 at the top of the container.

The container 4 is designed to cooperate with the test plate 1 by plugging it in.

A first needle 45 is provided on the test plate 1, each of which is arranged opposite the sealed inlet 41, and a second needle 46 is arranged opposite the sealed outlet 42.

When the container 4 is placed on the plate, the needles 45, 46 pierce the lids 64 and bring them into communication with each other:

the air inlet 14 with the inlet 41 and the stack 44, and on the other hand:
the outlet 42 with the biological sample inlet 12.

To empty the contents of the collection container 4, the station blows air into the air inlet 14, the air rises in the stack and pushes the biological sample towards the outlet 42 of the container, the biological sample liquid then flows from the inside of the container 4 into the inlet 12 of the plate. With this process, there is no risk of spilling biological sample liquid to the outside. Instead of using a positive relative pressure upstream, the same result is achieved by creating a vacuum downstream.

According to the present example, a plurality of single-use collection containers are used, each collection container is configured to be plugged onto a test plate, thus forming a pair [test plate+collection container] of single-use, thus intended to be discarded after use.

According to one embodiment of the collection container shown in FIG. 9B, the collection container 4ST comprises two pre-filled spaces for lysis and ethanol, respectively, and may be selectively placed in communication. More specifically, the container is provided with a volume of lysis in a main compartment and a volume of ethanol in an auxiliary compartment 47. The raw body fluid sample is then deposited into the main compartment with a sealable swab 49. The lid 43 is then closed tightly and the collection container 4ST is shaken. The lysis buffer breaks down the biological cell membranes. Then the annex compartment 47 is pierced to let the ethanol flow out so that it mixes with the lysed liquid. For this purpose a pointed or sharp element 48 is provided which may be moved by manual action on a button protected in a hole.

This forms a very practical collection kit.

In this fourth example, as seen in FIG. 10, the interfaces of the plate with the analysis station are on a first side, i.e. a first main face 10A and the inlet 12 for receiving a biological sample to be tested ET is arranged on the second side, i.e. on the second main face 10B.

Conversely, it may be seen that for the first three examples, all the interfaces of the plate to the analysis station are on one side 10A.

As shown in FIG. 11, other features are visible that may be independent of the configuration of the collection container. Here, the plate does not have an on-board valve. The selection or routing valves are located in the analysis station 8. In addition, the air pump works in vacuum instead of overpressure. It is ‘pulled’ instead of ‘pushed’.

Specifically, the plate has four air inlets, the previously commented upon inlet 14 for advancing the biological sample into the plate. Another inlet 142 is connected to the channel to which the first rinse volume 61, R1 arrives. Another inlet 143 is connected to the channel on which the second rinse volume 62, R2 arrives. A further inlet 144 is connected to the channel to which the first elution volume 63, 23 arrives. A special channel may be provided for drying in which case there is a fifth air inlet 145. Only one of the upstream solenoid valves 146 is open at a time, the others remain closed.

Downstream there are two air outlets, a first air outlet 16 at the bottom of the recovery reservoir 18 and a second air outlet 16b fluidly connected to the auxiliary reservoir 38. There may be an air outlet for drying, noted 16c, in fluid communication with the downstream housing 24 of the extraction membrane 2. Only one of the upstream solenoid valves 147 is open at a time, to generate a vacuum in one of the channel circuits.

To flush the diaphragm with the first flushing liquid, the second upstream valve and the downstream valve 1472 connected to output 16 are opened. To flush the diaphragm with the second flushing liquid, the third upstream valve is opened instead. To dry the diaphragm, the first downstream valve 1471 and the fifth upstream valve are opened.

For elution, the fourth upstream valve and the third downstream valve 1473 connected to the outlet are opened.

### Fifth Example of a Realization

FIGS. 17 to 22 illustrate a fifth embodiment.

The fifth example should be considered as a variation of the first example. For the elements not commented on in the following paragraphs, these elements should be considered to be the same or similar to what was described for the first example embodiment.

In general, some of the elements have different positions in this fifth example, but the basic scheme of the channels and housings remains similar.

As in the first example, the test plate has a generally flat parallelepiped shape, with two main faces, namely a first main face 10A, called the interaction face, containing all the interfaces, and a second main face 10B, called the rear face, containing no functional interface. The thickness of the board is between 2 mm and 8 mm. The length is between 30 mm and 80 mm (for example between 70 mm and 80 mm) and the width is between 10 mm and 60 mm (for example between 40 mm and 60 mm).

The sample to be tested ET is introduced through the inlet 12. A check valve is provided at this point.

The recovery reservoir 18 contains an absorbent pad 180. This absorbent pad has a blotting effect and captures liquids. The absorbent pad does not occupy the entire volume available in the recovery reservoir, an air passage between the pad and the walls of the reservoir allows air to escape through the outlet port 16.

A heating and reading device generally marked 120 is disposed opposite the first main face 10A of the plate. More specifically, as illustrated in FIG. 19, in the analysis position, the optical reading device faces the reading zones 31 32.

For heating, a first heating portion 96a is provided opposite the housing 24 of the extraction membrane 2 and a second heating portion 96 is provided opposite the reaction zones, in particular below the housings 72, 73.

The first heating portion 96a is provided to heat the extraction membrane (2) to a temperature of between 50° C. and 60° C. This step is noted as S30 in FIG. 12.

In other words, the first housing 24, the second housing 72 and the third housing 73 are arranged in close proximity to each other. The thermal interaction zone 126 covers the three aforementioned housings 24, 73, 73.

The thermal interaction zone 126 has an area of at most 4 cm² (or even 5 cm²). For example, the thermal interaction zone 126 may be 3 cm².

The heating and reading device may be equipped with one or more heating and cooling elements.

In this fifth example, a single flush fluid reservoir 61 is provided.

It is noted that in this fifth example, the functional interfaces, namely the inputs and the elements activated by the pushbuttons, are arranged on a single main face (10A). Furthermore, all functional interfaces, namely inputs and push-button activated elements, are grouped in an interface zone marked 190 in FIG. 22, on one side of the main face only, the interface zone 190 being located. The interface zone 190 is located within the cavity 87, and more specifically at the bottom of the cavity 87. It is noted that the complement of the interface zone 190 on the main face could be located at least partially outside the analysis station. In an example described above, this complement is a portion of the rigid support 39. In this way, a very compact analysis station may be designed.

Mirroring the thermal interaction zone of the plate, the analysis station 8 may also include a thermal interaction zone. This means that, in the cavity 87, the heating device 96 (especially the intermediate bodies), the optical analysis device are also included in an area that is less than 4 cm².

The optical transmitting and receiving cells 98, 99 are arranged on the heating and reading device 120. The components and method of optical reading are the same or similar to that described for the first example embodiment.

Moreover, it is noted that the auxiliary reservoir 38 interposed between the housings 72, 73 has here a diamond shape and not a circular shape. This allows the reaction discs to be flooded in an optimal manner.

### Various Other Aspects

In the first embodiment, as well as the 3rd, as well as the 4th, as well as the 5th, the test plate is monolithic and contains in a single body the following elements: the extraction membrane, one or two reaction zones, the volumes of rinse liquid and eluent, the recovery reservoir, and the result reading zone.

It is noted that it is possible to use the particular collection containers 4, 4ST described in the fourth example in the context and implementation of the other examples.

It should be noted that there is no electrical source on board the plate, only pre-stored chemical compounds and reagents in freeze-dried form and liquids where appropriate.

The overpressure or underpressure created by the air pump 85, relative to atmospheric pressure, may be between 100 mbar and 500 mbar. This overpressure or negative pressure may be adapted according to the portion of the cycle (rinsing, elution, drying).

It is noted that ribs 19 may be provided in the recovery reservoir, thereby controlling the advance of the liquid front into the recovery reservoir. The ribs 19 are located at the bottom of the recovery reservoir when the plate is in the test position on station 8, the grooves 19 form mini dams which provide hydrodynamic resistance to the advance of liquids into recovery reservoir 18.

It is noted that it may be provided that the recovery reservoir 18 is a single plate edge recovery reservoir, said recovery reservoir 18 being configured to collect all liquids and trap them within the plate. Nothing comes out of the plate. Only air can emerge from the plate. This prevents contamination of the environment and/or the next test.

Using a wireless link 196, the analysis station interfaces with a smartphone or tablet 28.

In addition, the analysis station may interface with a server 29.

Advantageously, the analysis station is updated by downloading. In particular, the settings and certain parts of the software may be updated by downloading from a server.

For the actuators 91-95 arranged in the station, linear actuators are used. For example, one may choose from:

a pneumatic system cylinder which directly activates a piston intended to push on the membrane or the volume of liquid to be mechanically stressed,
an electric cylinder or stepper motor with a rack and pinion ora screw and nut mechanism
a solenoid or electromagnet
a piezoelectric stage stack, especially for membrane valves.

Although the description primarily cites the LAMP (Loop-mediated isothermal amplification) or RT-LAMP (Reverse transcriptase Loop-mediated isothermal amplification) method, this biological test system and its inserts may be used for the following methods:

RPA: Recombinase polymerase amplification, or RT-RPA,

HDA: Helicase dependent amplification, or RT-HDA,

RCA: Rolling circle amplification, or RT-RCA,

SDA: Strand displacement amplification, or RT-SDA,

NASBA: Nucleic acid sequence-based amplification, or RT-NASBA,

TMA: Transcription mediated amplification, or RT-TMA.

1. A test plate, for single use in a biological test system for detecting the presence of one or more pathogenic species, the test plate comprising:
a body in which channels and housings are formed, which are generally isolated from the external environment,
a sample inlet for receiving a biological sample to be tested in liquid form;
at least one air inlet;
an extraction membrane arranged in a first housing;
a first reaction zone containing a porous media arranged in a second housing and containing a first test reaction mixture in lyophilized form, the first reaction mixture allowing the amplification and detection of at least one nucleic acid sequence of interest;
at least one air outlet;
a recovery reservoir, and
a result reading zone; comprising at least the first reaction zone and, if applicable, a second reaction zone, and
the test plate being configured to feed the biological sample to be tested into the extraction membrane, and then, after rinsing, drying and eluting said extraction membrane, to feed the resulting eluate into at least the first reaction zone and, if applicable, the second reaction zone, in order, after reaction, to deduce a result through the reading zone.

2. The test plate according to claim 1, further comprising at least a first volume of first rinse liquid and a volume of eluent, contained directly in the housings of the body or contained in pouches arranged in the housings of the body.

3. The test plate according to claim 2, wherein the eluent contained in the eluent volume is distilled water free of RNAase and/or DNAase and the eluent volume has a volume between 30 microliters and 50 microliters.

4. The test plate according to claim 2, further comprising a second volume of a second rinse liquid, the first and second volumes of rinse liquid having a volume of between 100 microliters and 300 microliters.

5. The test plate according to claim 1, further comprising at least two valves, for directing a flow of fluid from the extraction membrane selectively to the recovery reservoir or to the first reaction zone and, if applicable, the second reaction zone.

6. The test plate according to claim 1, further comprising an auxiliary reservoir arranged adjacent to the first, reaction zone so as to be able to supply eluate to the first and second reaction zone by capillary pumping.

7. (canceled)

8. The test plate according to claim 1, further comprising a membrane or vent valve, arranged on at least one of the at least one air outlet to let air pass to the atmosphere and not to let liquid pass.

9. The test plate according to claim 1, wherein an intermediate air outlet is provided arranged near an inlet of the recovery reservoir, to facilitate drying of the membrane after the passage of the rinse liquids.

10. The test plate according to claim 1, having a generally flat parallelepiped shape, with two main faces with the at least one air inlet and the sample inlet for receiving a biological sample to be tested are arranged on one main face.

11. (canceled)

12. The test plate according to claim 1, wherein the test plated is configured to receive, by plugging in, a collection container containing a biological sample to be tested.

13. (canceled)

14. (canceled)

15. The test plate according to claim 1, wherein the first housing and the second housing are arranged in proximity to each other, preferably in a thermal interaction zone of at most 4 cm².

16. The test plate according to claim 1, comprising two main faces, opposite each other, and all functional interfaces for testing are located on only one of the two main faces.

17. (canceled)

18. An assembly comprising a test plate according to claim 16, and a rigid support on which the test plate is mounted, the rigid support integrally covering the main face not having the functional interfaces.

19. (canceled)

20. A test equipment comprising a test plate according to claim 12, and a collection container comprising a sealed inlet and a sealed outlet, both arranged at a bottom of the collection container, and a tightly closing lid.

21. The test equipment according to claim 20, wherein the collection container is plugged onto the test plate, and wherein the test plate comprises two needles arranged opposite the sealed inlet and the sealed outlet respectively, the two needles passing through caps during the plugging operation.

22. A biological testing system, comprising:
an analysis station, and
one or more test plates according to claim 1, the analysis station comprising at least one pneumatic connector configured to be coupled to the air inlet and/or the air outlet.

23. The biological testing system according to claim 22, wherein the analysis station comprises at least one of the following:
a control unit,
an air pump,
one or more actuators; to push on volumes and/or activate controlled valves,
an optical analysis device to determine the result,
a heating device,
a wireless communication system,
an identifier reader, and
a cover locking system and, a Hall effect sensor for detecting the closing of the cover.

24. The biological testing system according to claim 23, the analysis station comprising:
a base configured to receive the test plate,
wherein the base comprises a cavity complementary in shape to a main face of the test plate, such that the test plate is snappable into the cavity and removed by exerting a force in opposite direction to the cavity,
wherein the cavity comprises an interface area for the air pump, the one or more actuators, the optical analysis device and the heating device, the interface area being configured to cooperate with a functional interface of the main face of the cartridge.

25. The biological testing system according to claim 24, wherein the optical analysis device and the heating device are arranged in an area of the cavity close to each other.

26. The biological testing system according to claim 24, comprising a cover, rotatably mounted with respect to the baseplate, and configured to, in an open position, allow an operator to engage and disengage the test plate in the cavity and, in a closed position, to plate the plate in the cavity.

27. (canceled)

28. A biological test process implemented in a test plate according to claim 1, the method comprising:
passing the biological sample to be tested in liquid form through the extraction membrane, and direct it the biological sample downstream to the recovery reservoir,
passing a first rinse liquid through the extraction membrane, and directing it the first rinse liquid downstream to the recovery reservoir,
passing air through the extraction membrane to dry it the extraction membrane,
passing a volume of eluent through the extraction membrane, and directing an eluate which emerges downstream towards the first reaction zone.

29. (canceled)

30. (canceled)

31. (canceled)

32. (canceled)

33. The test plate according to claim 1, further comprising the second reaction zone, the second reaction zone containing a porous media arranged in a third housing and containing a second control reaction mixture in lyophilized form.

34. The test plate according to claim 5, wherein each valve of the at least two valves is of the membrane type.
